(19) United States
(12) Patent Application Publication
(10) Pub. No.: US 2025/0387219 A1
(43) Pub. Date: Dec. 25, 2025

(54) FLUID FOR ACCOMMODATING INTRAOCULAR LENSES

(71) Applicant: Alcon Inc., Fribourg (CH)

(72) Inventors: David J. ANVAR, Sunnyvale, CA (US); Andrew GOODWIN, San Leandro, CA (US); David CHAZAN, Palo Alto, CA (US)

(73) Assignee: Alcon Inc., Fribourg (CH)

(21) Appl. No.: 19/309,360

(22) Filed: Aug. 25, 2025

Related U.S. Application Data
(62) Division of application No. 18/348,696, filed on Jul. 7, 2023, now Pat. No. 12,419,738.
Application No. 18/348,696 is a division of application No. 17/200,047, filed on Mar. 12, 2021, now Pat. No. 11,737,862.
Application No. 17/200,047 is a continuation of application No. 14/555,001, filed on Nov. 26, 2014, now Pat. No. 10,980,629.
Application No. 14/555,001 is a continuation of application No. 13/033,474, filed on Feb. 23, 2011, now Pat. No. 8,900,298.
(60) Provisional application No. 61/307,354, filed on Feb. 23, 2010.

(51) Int. Cl. A61F 2/16; A61L 27/14; A61L 27/50; C07F 7/18; C07F 7/20; C08G 77/00; C08G 77/34; C08L 83/04

(52) U.S. Cl. CPC A61F 2/1613; A61L 27/14; A61L 27/50; C07F 7/1804; C07F 7/20; C08G 77/34; C08G 77/80; C08L 83/04

(57) ABSTRACT

Fluids incorporated into intraocular lenses and their methods of use. In some embodiments the fluids are silicone oils, and in some embodiments they are used in accommodating intraocular lenses.

### CROSS-REFERENCE TO RELATED APPLICATIONS

This application is a divisional of U.S. application Ser. No. 18/348,696, filed Jul. 7, 2023, which is a divisional of U.S. application Ser. No. 17/200,047, filed Mar. 12, 2021, now U.S. Pat. No. 11,737,862, which is a continuation of U.S. application Ser. No. 14/555,001, filed Nov. 26, 2014, now U.S. Pat. No. 10,980,629, which is a continuation of U.S. application Ser. No. 13/033,474, filed Feb. 23, 2011, now U.S. Pat. No. 8,900,298, which application claims the benefit of U.S. Provisional Patent Application No. 61/307,354, filed Feb. 23, 2010, the disclosures of which are incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Intraocular lenses (“IOL”) may comprise one or more fluids disposed therein. For example, some accommodating IOLs use fluid movement within the IOL, or a change in fluid pressure within the IOL, to effect optical power change in the IOL. Exemplary accommodating IOLs that include a fluid can be found in U.S. Pat. App. Pub. Nos. 2008/0306588, filed Jul. 22, 2008, and 2008/0306587, filed Jul. 22, 2008, the disclosures of which are incorporated herein by reference. Exemplary methods of accommodation in response to natural ciliary muscle movement are also described therein. For example, in the embodiment shown in FIGS. 3-5 in U.S. Pat. App. Pub. No. 2008/0306588, a fluid pressure increase in the optic portion causes the shape of the anterior surface of the optic portion to change, thereby changing the power of the lens. Silicone oil is an example of a fluid that can be used in an IOL. In the embodiment shown, the peripheral portion is in fluid communication with the optic portion, allowing, for example, silicone oil to flow between the optic portion and the peripheral portion. The bulk material of the lens includes anterior lens element 16, intermediate layer 18, and posterior element 22. The bulk material can also be considered to include the haptic bulk material in the peripheral portion of the IOL.

When fluids such as silicone oil are used in an accommodating intraocular lens, the fluid, over time, may tend to swell into the bulk material. This can reduce the amount of silicone oil available to drive the optical power change in the IOL. It is therefore desirable to minimize the amount of swelling into the bulk material. It may also be important to provide silicone oil that does not reduce the response time of the accommodating IOL.

Some IOLs rely on, or can benefit from, a substantially uniform refractive index throughout the IOL. It may therefore also be beneficial to provide silicone oil that has a refractive index that is as close to the refractive index of the bulk material as possible.

Improved fluids (e.g., silicone oils), their methods of manufacture, and their methods of use in accommodating intraocular lenses are therefore needed.

### SUMMARY

One aspect of the disclosure is a method of manufacturing silicone oil for use in an intraocular lens, comprising purifying silicone oil to be used in an intraocular lens, wherein the silicone oil has a polydispersity index of less than about 1.5, and in some embodiments less than about 1.3. The silicone oil can have a mean molecular weight of between about 5000 Daltons and about 6500 Daltons. In some embodiments there is no more than about 50 ppm of any low molecular weight component, such as components that have a molecular weight of about 1000 Daltons or less, in the silicone oil to be used in the intraocular lens. In some embodiments the method includes controlling the refractive index of the silicone oil to be between about 1.47 and about 1.49. In some embodiments purification step is a supercritical CO₂ extraction, while in some embodiments it is a wiped-film extraction. The purification step substantially prevents the silicone oil from swelling in a bulk polymeric material of the intraocular lens. In some embodiments the silicone oil comprises diphenyl siloxane and dimethyl siloxane, and in some particular embodiments there is about 20% diphenyl siloxane and about 80% dimethyl siloxane.

One aspect of the disclosure is a method of manufacturing silicone oil for use in an intraocular lens, comprising synthesizing silicone oil to be used in an intraocular lens, wherein the silicone oil has a polydispersity index of less than about 1.5. The synthesis can be a living polymerization synthesis. The method also includes a purification step after the synthesis step, which can be, for example, a supercritical CO₂ extraction or a wiped-film purification step. In some embodiments the silicone oil has a mean molecular weight of between about 5000 Daltons and about 6500 Daltons. In some embodiments there is no more than about 50 ppm of any low molecular weight component in the silicone oil. In some embodiments the viscosity of the silicone oil is less than about 1000 cSt at about 25° C.

One aspect of the disclosure is a method of manufacturing silicone oil for use in an intraocular lens, comprising purifying silicone oil to be used in an intraocular lens, wherein the silicone oil has a mean molecular weight between about 5000 Daltons and about 6500 Daltons. The silicone oil can have a polydispersity index of less than about 1.5. In some embodiments there is no more than about 50 ppm of any low molecular weight component in the silicone oil. The manufactured silicone oil is adapted to avoid swelling in a bulk polymeric material of the intraocular lens. The silicone oil can comprise diphenyl siloxane and dimethyl siloxane.

One aspect of the disclosure is a method of manufacturing an intraocular lens, comprising providing a silicone oil that has been purified to have a polydispersity index of less than about 1.5; and assembling a bulk polymer material and the silicone oil to form an intraocular lens. The assembling step can comprise advancing the silicone oil into a fluid chamber within the bulk material of the intraocular lens. The silicone oil can have been purified to have a mean molecular weight between about 5000 Daltons and about 6500 Daltons. The silicone oil can have been purified such that there is no more than 50 ppm of any component that has a molecular weight of about 1000 Daltons or less. In some embodiments the silicone oil has been substantially index-matched to at least a portion of the bulk material.

One aspect of the disclosure is a method of using an intraocular lens: comprising creating an opening in the eye; and implanting in a posterior chamber of an eye an intraocular lens comprising silicone oil purified to have a polydispersity index of less than about 1.5.

One aspect of the disclosure is silicone oil adapted to be used in an intraocular lens, wherein the silicone oil has been purified and has a polydispersity index less than about 1.5. The silicone oil can comprise diphenyl siloxane and dimethyl siloxane, and in some embodiments the silicone oil comprises about 20 mol % diphenyl siloxane and about 80% dimethyl siloxane. The silicone oil can have a mean molecular weight between about 5000 Daltons and about 6500 Daltons, and there are no more than 50 ppm of any component that has a molecular weight of about 1000 Daltons or less. In some embodiments the silicone oil has a viscosity of less than about 1000 cSt at about 25° C. The refractive index can be between about 1.47 and about 1.49.

One aspect of the disclosure is silicone oil adapted to be used in an intraocular lens, wherein the silicone oil has been synthesized and has a polydispersity index less than about 1.5.

One aspect of the disclosure is an accommodating intraocular lens comprising a bulk polymeric material and silicone oil that has a polydispersity index less than about 1.5. The silicone oil can have an index of refraction between about 1.47 and about 1.49. The silicone oil can comprise diphenyl siloxane and dimethyl siloxane. The silicone oil can have a mean molecular weight number average of between about 5000 Daltons to about 6500 Daltons. The viscosity of the oil can be less than about 1000 cSt at about 25° C. In some embodiments there is no more than 50 ppm of any component that has a molecular weight of about 1000 Daltons or less.

### DETAILED DESCRIPTION

The disclosure herein generally relates to fluid, such as silicone oil, that is used in an intraocular lens. In some embodiments the silicone oil is used in an accommodating intraocular lens that uses fluid movement to effect optical power change in the IOL. The silicone oil can, however, be used in non-accommodating intraocular lenses as well.

Accommodating IOLs can utilize the eye's natural ciliary muscle movements to provide accommodation in the IOL. For example, some accommodating IOLs are implanted within a patient's capsular bag (after the native lens has been removed) and respond to capsular bag reshaping to change the power of the lens. Some IOLs are designed to be implanted outside of the lens capsule and accommodate in other ways. Whatever the method of accommodation, silicone oil disposed within an accommodating IOL can be adapted to be moved within the IOL in response to the eye's natural movement in order to change the lens power. Properties of the silicone oil can therefore affect the accommodative response time of the IOL. The selected silicone oil therefore does not undesirably hinder the response time of the IOL.

When silicone oil is used in accommodating IOL with a bulk material such as a polymeric material, some of the oil components can pass into the bulk material, causing the bulk material to swell. The selected silicone oil or oils therefore avoids the undesirable swelling of the bulk polymer. Exemplary polymeric materials that can be used for the bulk material of the IOL can be found in U.S. application Ser. No. 12/177,720, filed Jul. 22, 2008, and in U.S. application Ser. No. 12/034,942, filed Feb. 21, 2008, the disclosures of which are incorporated herein by reference.

One characteristic of silicone oil that helps ensure an adequate response and avoids undesirable swelling is the polydispersity index (“PDI”) of the silicone oil to be used in the IOL. PDI is generally a measure of the distribution of molecular mass in a given sample. A relatively low PDI indicates a relatively narrow range of molecular weights. The silicone oils described herein have a PDI less than about 1.5, and more particularly less than or equal to about 1.3.

A second characteristic of the silicone oil that helps ensure an adequate response and avoids undesirable swelling is the mean molecular weight of the silicone oil. When high concentrations of relatively low molecular weight components are present in the silicone oil, a greater number of low molecular weight components pass into the bulk material of the IOL causing the swelling of the bulk material. To avoid undesirable swelling, the concentration of relatively low molecular weight components should be minimized. By reducing the concentration of relatively low molecular weight components and maintaining a high concentration of relatively high molecular weight components, fewer low molecular weight components will pass into the bulk polymer material, reducing the amount of swelling that occurs in the bulk material.

The PDI of the silicone oil and the mean molecular weight of the oil are related—by lowering the PDI of the silicone oil while providing silicone oil with high concentrations of relatively high molecular weight components and low concentrations of low molecular weight components, the response of the IOL is maintained (by providing a silicone oil with suitable viscosity) and undesirable swelling is avoided. Additionally, providing silicone oil with a low PDI and very low concentrations of small molecular weight components means that the silicone oil has a molecular weight just large enough to avoid swelling of the polymer.

In some embodiments silicone oil is provided that has a mean molecular weight between about 5000 and about 6500 Daltons, which is large enough to substantially avoid swelling of the bulk polymeric material. This is preferable to the alternative, which is using a higher molecular weight silicone oil which has inherently fewer small molecule components because almost all molecules comprising it are large. High molecular weight silicone oils can have a correspondingly high viscosity, which can reduce the response time of the accommodating IOL.

The silicone oils described herein have a very low concentration of relatively low molecular weight components. The very low molecular weight components are present in an amount less than about 200 ppm of each component, and in some embodiments less than about 100 ppm. In some particular embodiments the very low molecular weight components are present in an amount less than about 50 ppm.

The relatively low molecular weight components include those less than or equal to about 1000 Daltons. For example, in some embodiments the concentration of components less than or equal to about 1000 Daltons is not more than about 50 ppm.

In one particular embodiment, silicone oil is provided in which no more than 20% of the total silicone by weight is comprised of components below about 4000 Daltons; no more than 10% of the total polymer fluid by weight is comprised of components below 3000 Daltons; and no more than 50 ppm of any components below 1000 Daltons.

The estimated molecular weights and polydispersities described herein are relative to polystyrene molecular weights standards.

The silicone oil generally needs to be designed in such a way as to avoid adverse interactions with the surrounding bulk IOL material, such as swelling, fogging, dissolving or reacting with the material (e.g., poly acrylate) in some IOLs. The degree of solubility of the silicone oil in the bulk material is dependent on the chemical structure and molecular weight distribution of the silicone oil. Other parameters that influence this interaction are the composition and properties of the bulk material such as homogeneity, chemical structure, hydrophobicity, modulus, and crosslink density.

The viscosity of the silicone oil also generally needs to be defined and minimized because, in embodiments in which the fluid-driven accommodating IOL operates dynamically, the IOL must have an appropriate response time. In some embodiments the viscosity of the silicone oil is less than about 1000 cSt at 25° C.

In some embodiments the silicone oil is comprised of diphenyl siloxane and dimethyl siloxane. In some embodiments the oil is a diphenyl siloxane and dimethyl siloxane copolymer with about 20 mol % diphenyl siloxane and about 80 mol % dimethyl siloxane.

In some IOLs it may be desirable to avoid creating an optical interface between the bulk material of the IOL and the silicone oil within the IOL. This can be done by index-matching the silicone oil to the bulk material of the IOL, which in some embodiments is a polymeric material. “Index-matching” as used herein refers to minimizing the optical interface between first and second media. For example, index-matching silicone oil and a polymeric material refers to attempting to eliminate an optical interface therebetween, and “substantially the same” refers to indexes of refraction that, even though they may be slightly different, are intended to be as close as possible to minimize the difference in refractive indexes.

In some embodiments in which the silicone oil is index-matched to the bulk polymeric material, the refractive index of silicone oil is between about 1.47 and about 1.53, and in some embodiments is between about 1.47 and about 1.49.

In some embodiments the silicone oil must be able to be filtered through an about. 7 micron filter. In some embodiments the percent volatiles are less than about 0.2%. In some embodiments the silicone oil has a chromatic dispersion less than or equal to about 0.035 refractive index units in the visible range of 400 nm to 750 nm at 35° C. In some embodiments the silicone oil components are fully miscible with each other without evidence of phase separation (i.e. cloudiness or suspensions). In some embodiments the silicone oil has greater than 85% transmittance in the range of 400 nm to 1100 nm for about a 1 cm thick fluid sample.

In addition, the silicone oil should be clear, colorless, have less than about 10 ppm heavy metals and other insoluble inorganics contaminants, and have substantially no silanols.

### Synthesis

The molecular weight, polydispersity, and in some instances the refractive index of the silicone oil can be controlled by the way in which the silicone oil is synthesized and purified. The viscosity of the oil is related to the molecular weight of the oil, the polydispersity of the oil, and the architecture of the bulk polymer, all of which are influenced by the synthesis and purification of the polymer. However, a target viscosity can not be arbitrarily selected independent of the target molecular weight, polydispersity, composition, and architecture of the silicone oil. A general class of polymer synthesis reactions known as “living polymerization reactions” can offer the degree of control necessary to assist in meeting some of the design requirements for a silicone oil.

The term “living polymerization” implies a polymerization reaction that does not have a significant number of chain terminating or chain transferring side reactions. The absence of side reactions allows living polymerizations to be used to synthesize a variety of materials that would be otherwise difficult to prepare. This class of polymerization reactions can be used to prepare polymers with a variety of 1) architectures-including linear, “star”, and “comb” polymers; 2) compositions-homopolymers, random copolymers, block copolymers, and graft copolymers; and 3) functionalized polymers-one and two end functional polymers, and side functional polymers. This class of polymerization reactions can be used to prepare polymers that often have a narrow molecular weight distribution and at a variety of molecular weights. As a result, living polymerizations are often employed when polymers with specific structures and compositions are needed. For example, a polymer with a large molecular weight distribution can be considered to be a mixture of a large number of compounds, and the properties of the material are some function of that distribution. Polymers that have a small molecular weight distribution, however, as can result from a living polymerization, can be considered a “purer” sample, with properties that are better defined.

Anionic and cationic living polymerizations have been described in the art. More recently, radical living polymerizations may have been developed. In an example of an anionic synthetic route, the use of alkyl lithium compounds in the ring opening polymerization of cyclotrisiloxanes appears to be a “living” polymerization, allowing for the degree of control needed to make the silicone oils described above. By varying the ratio of phenyl containing cyclotrisiloxanes to methyl only containing cyclotrisiloxanes (that is, preparing a random block copolymer), the refractive index of the silicone oil can be varied between the refractive index of either pure homopolymer alone (i.e., between pure diphenyl polysiloxane and pure dimethyl polysiloxane).

As another example, the refractive index of the silicone oil can be varied by varying the ratio of a tetramethyl-diphenyl-cyclotrisiloxane to hexamethyl cyclotrisiloxanes. Varying this ratio can provide different refractive indexes between about 1.40 and about 1.54, including those between about 1.47 and 1.49.

As mentioned above, a living polymerization also offers the advantage of being able to prepare polymer products of a targeted molecular weight. This can be accomplished by varying the monomer to initiator ratio during the polymerization reaction, an application which can be applied to the preparation of silicone oils of a specified formula weight.

The feature of a narrow range of molecular weight products is also an advantage that can be realized in the preparation of silicone oils because fewer low molecular weight oligomers are made during the polymerization reaction. The smaller quantity of the low molecular weight materials prepared minimizes the amount of purification that needs to occur later to remove them from the higher molecular weight products. For example, when fewer low molecular weight oligomers are made during the polymerization reaction, it is easier to extract the low molecular weight materials when purifying the synthesized silicone oil using a supercritical CO₂ extraction (described below), resulting in higher yields of the desired product.

While the viscosity of a polymer is not directly related to the way in which the polymer is prepared, a living polymerization can also be used to indirectly modify this feature of the product polymer. Living polymerizations can be used to make polymer architectures that would be difficult to accomplish using other synthetic strategies. For example, “comb” polymers, “star” polymers, and other branched structures can be prepared, which, even though they have a very similar chemical composition to a “linear” polymer, may have different physical properties (e.g., viscosity), because of the different physical geometries those structures have. Preparation of a highly branched silicone oil may yield a product which has a significantly lower viscosity than a silicone oil with the same molecular weight but a linear structure.

Silicone oils can also be prepared using other synthetic strategies such as the base catalyzed ring opening of cyclotrisiloxanes, and the condensation of dialkyldichloro silanes with water. These synthetic strategies can also prepare silicone oils with many of the characteristics described above, but can require more effort on purification.

### Purification

Silicon oils can be purified in a variety of ways. Wiped film evaporation can be used to remove low molecular weight compounds that have a high boiling point. The silicone oil product may, however, be discolored on excessive heating when using wiped film evaporation.

Supercritical CO₂ extraction is one exemplary purification method that can be used to selectively remove fractions of silicone oil based on molecular weight and based on chemical affinity. Supercritical CO₂ extraction to purify silicone oils to produce silicone vitreoretinal tamponades is described in U.S. Pat. No. 7,276,619, the entire disclosure of which is incorporated by reference herein. These oils are not used for IOLs, are particularly not in fluid-drive accommodating IOLs. Pressure, temperature, rate of extraction conditions, and the use of co-eluting solvents such as, for example, acetone, can be varied to yield fractions that have a narrow molecular weight distribution (i.e., a low PDI). A mixture can be separated in such a way as to strip the very low and very high molecular fractions from a sample achieving the desired molecular weight. Because supercritical extraction conditions can be varied to get separation based on chemical affinity, this purification method can also be used to achieve a desired refractive index. Supercritical CO₂ extraction can therefore be used to produce a silicone oil with, for example, an index of refraction substantially the same as a bulk polymer to be used in an intraocular lens (e.g., in a fluid-driven accommodating intraocular lens).

Tables 1-3 provide data from exemplary supercritical CO₂ extractions of sample silicone oils.

TABLE 1

| Silicone Oil Sample | Time at 85 C. (Hrs) | % Weight Change |
|---|---|---|
| 1 | 404 | 43.15 |
| 2 | 404 | 24.48 |
| 3 | 404 | 11.11 |
| 4 | 404 | 6.15 |
| 6 | 404 | 1.67 |
| 7 | 404 | 13.25 |

TABLE 2

| Silicone Oil Sample | Mean RI |
|---|---|
| 1 | 1.477792 |
| 2 | 1.48604 |
| 3 | 1.487633 |
| 4 | 1.49067 |
| 5 | 1.494362 |
| 6 | 1.498737 |
| 7 | 1.492858 |

TABLE 3

| Silicone Oil Sample | Viscosity (cP) at 25.0 C. | stdev |
|---|---|---|
| 1 | 38.40 | 1.20 |
| 2 | 87.12 | 1.37 |
| 3 | 175.68 | 2.01 |

Similarly, preparative scale size exclusion chromatography is an alternative method to fractionate a polymer sample into molecular weight components. Fractional precipitation of the silicone oil may also be used to separate components of the product polymer.

Removal of silicone oil components that dissolve into the bulk IOL material over time (e.g., during storage) may also be accomplished by exposing the silicone oil to bulk quantities of the IOL material, or other materials that have been selected for that purpose. On storage with an appropriate material, the components of the silicone oil that dissolve into the bulk IOL polymeric material may be removed by adjusting the ratio of silicone oil to polymer adsorbent so that sufficiently low levels of those materials remain in the oil.

While silicone oils used in accommodating IOLs are primary described herein, it is possible to use any of the silicone oils in a non-accommodating IOL. For example, a non-accommodating IOL can have a relatively rigid outer polymeric shell surrounding a silicone oil core. Swelling of the bulk polymeric material would still need to be taken into consideration, and hence the methods of manufacturing desired silicone oil described herein could be utilized.

What is claimed is:

1. A method of manufacturing an intraocular lens, comprising:
assembling the intraocular lens using in part a bulk polymeric material; and
introducing a silicone oil into a fluid chamber defined within the intraocular lens, wherein the silicone oil has a polydispersity index of less than 1.5.

2. The method of claim 1, further comprising synthesizing and purifying the silicone oil to be introduced into the fluid chamber of the intraocular lens.

3. The method of claim 2, wherein synthesizing the silicone oil further comprises synthesizing the silicone oil using a living polymerization reaction.

4. The method of claim 2, wherein purifying the silicone oil further comprises purifying the silicone oil using supercritical CO₂ extraction.

5. The method of claim 1, wherein the intraocular lens is a fluid-driven accommodating intraocular lens.

6. The method of claim 1, wherein the silicone oil has a mean molecular weight of between 5000 Daltons and 6500 Daltons.

7. The method of claim 1, wherein a refractive index of the silicone oil is substantially the same as the refractive index of the bulk polymeric material, wherein the intraocular lens further comprises an optic portion, and wherein the optic portion is made in part of the bulk polymeric material, wherein the fluid chamber is within the optic portion.

8. A method of manufacturing a silicone oil for use in an intraocular lens, comprising:
synthesizing the silicone oil to be used in the intraocular lens using a living polymerization reaction; and
purifying the silicone oil.

9. The method of claim 8, wherein the silicone oil is synthesized using at least one of an anionic and a cationic living polymerization reaction.

10. The method of claim 8, wherein the silicone oil is synthesized as branched polymeric structures.

11. The method of claim 10, wherein the branched polymeric structures comprises a star-like polymeric structure.

12. The method of claim 10, wherein the branched polymeric structures comprises a comb-like polymeric structure.

13. The method of claim 10, wherein the branched polymeric structures comprises an X-shaped polymeric structure.

14. The method of claim 8, wherein synthesizing the silicone oil further comprises synthesizing the silicone oil using a base catalyzed ring opening reaction.

15. The method of claim 8, wherein synthesizing the silicone oil further comprises synthesizing the silicone oil using condensation of dialkyldichloro silanes with water.

16. The method of claim 8, wherein purifying the silicone oil further comprises purifying the silicone oil using supercritical CO₂ extraction.

17. The method of claim 8, wherein purifying the silicone oil further comprises purifying the silicone oil using wiped-film extraction.

18. The method of claim 8, further comprising purifying the silicone oil until the silicone oil has a mean molecular weight of between 5000 Daltons and 6500 Daltons.

19. The method of claim 8, further comprising purifying the silicone oil until no more than 20 wt % of the silicone oil is comprised of components below 4000 Daltons.

20. The method of claim 8, further comprising purifying the silicone oil until no more than 10 wt % of the silicone oil is comprised of components below 3000 Daltons.
